(19) United States
(12) Patent Application Publication
(10) Pub. No.: US 2017/0056684 A1
(43) Pub. Date: Mar. 2, 2017

(54) LIPOLYSIS EXERCISE DEVICE.

(71) Applicant: SASSON ELIHUE MOULAVI, BOCA RATON, FL (US)

(72) Inventors: ZBIGNIEW STEIN, PORT CHARLOTTE, FL (US); SASSON ELIHUE MOULAVI, BOCA RATON, FL (US)

(21) Appl. No.: 14/840,873

(22) Filed: Aug. 31, 2015

(51) Int. Cl. A61N 5/06

(52) U.S. Cl. CPC A61N 5/0613; A61N 2005/0626; A61N 2005/0653; A61N 2005/0651; A61N 2005/0663

(57) ABSTRACT

Lipolysis exercise device with the portability option and integrated tamper resistant hour counter for the device rental purpose.

### BACKGROUND

Field of Invention

The present invention relates to the lipolysis exercise device with the portability option and integrated hour counter for the device rental purpose.

### FEDERALLY SPONSORED RESEARCH

None

### SEQUENCE LISTING

None

Description of the Prior Art

At the present time lipolysis devices are a stationary clinical units requiring a wall plugged power supply. The present invention uses the option of the battery operated power supply in order to provide portability.

Also none of presently available devices comprises an hour counter. The present invention contains the tamper resistant hour counter being an integrated part of the liployzis device making it suitable for the rental purpose.

### SUMMARY OF INVENTION

The present invention relates to the lipolysis exercise device with the portability option and integrated tamper resistant hour counter for the device rental purpose.

### Objectives and Advantages

At the present time lipolysis devices are a stationary units used as a clinical equipment requiring a wall plugged power supply. The present invention uses the option of the battery operated power supply in order to provide portability and the possibility to use the equipment during the exercise. This is very important since the fat cells released to the blood stream may be burned right away during the exercise, which is much more effective than after the clinical lipolysis session. In the case when the exercise is not done or done too late after the lipolysis session the fat cells would be absorbed back by the body making the lipolysis session un effective.

The present invention also due to an hour counter being an integrated part of the lipolysis device may be widely used in gyms and fitness centers as a rental equipment. In this case the counter person, who rents the equipment direct to the users and collects a fee for each rental session is provided with the totalized sum of the usage time according to the readout of the integrated and prevented from tampering hour counter. This totalized sum is the base of the later financial reconciliations between the counter person and the lipolysis exercise device owner.

### BRIEF DESCRIPTION OF THE DRAWINGS

FIG. 1 shows the exercise lipolysis device with the electromechanical control system.

FIG. 2 shows the lipolysis device with microprocessor control system.

FIG. 3 shows the lipolysis exercise device microprocessor program flow chart.

### DESCRIPTION OF THE EMBODIMENTS

Herein, there follows the description of the present invention in its embodiments.

FIG. 1 shows the lipolysis exercise device 11 with the electromechanical control system solution containing the electromechanical timers 12 and 13, the wall plugged power supply 14, the battery operated power supply 15 that may be used sparingly with the power supply 14. The regulator 16 provides the voltage on the required level to power the multitude of the LED (Light emitting diodes) pads 19 through timers 12 and 13. Pads 19 energized by the regulator 16 provide the red light radiation of 635 nm wavelength required for the lipolysis process. The hour counter 10 counts the total time of the effective operation of the unit 11 with the LED pads 19 being switched on. Both timers 12 and 13 are equipped with the LED test lamps 17 and 18. This lamps are on when the timers are off and are switched off when the timers are on, thus providing the simple test for the timers. The timers are meant to be set for the same time period of max. 20 min. and the session would end as soon as any of them expires. This configuration would not allow the session to last longer in case of any of this timers failure. The LED test lamps allow to test both timers before each session and in case of the failure of one of them the equipment shouldn't be used until the failed timer would be replaced. The master timer 12 is not accessible by the patient while the secondary timer may be observed by the patient and would give him the readout of the session time left. The session time can't be extended by the patient, since in order to do it both timers has to be set for the extended session time.

Herein, there follows the description of the present invention in its second embodiment with additional possible options.

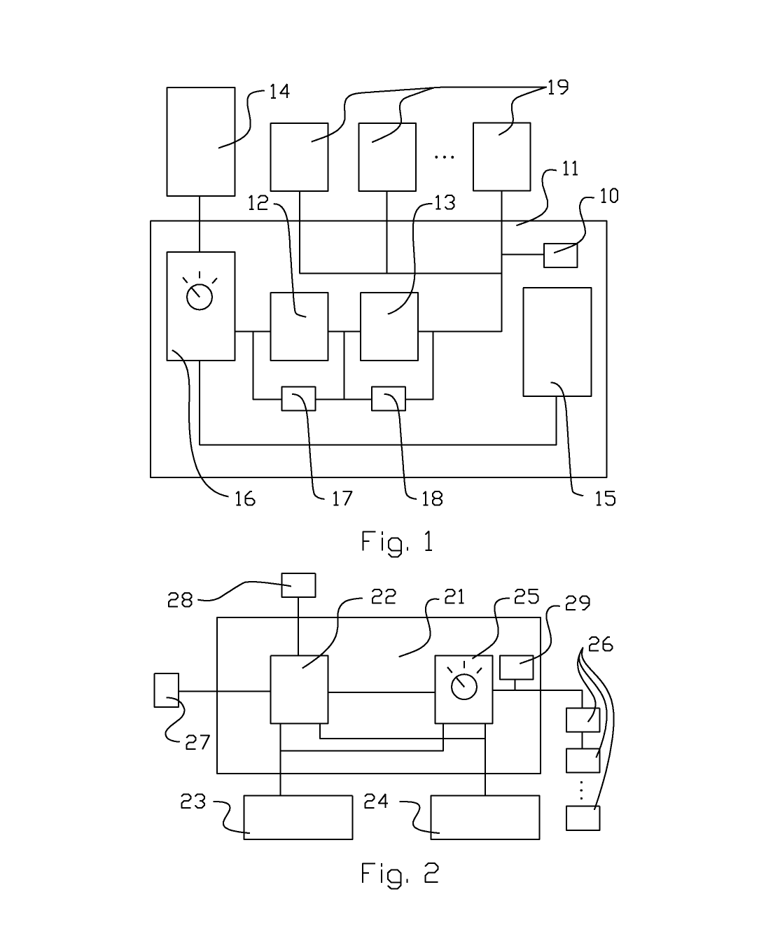

FIG. 2 shows the lipolysis exercise device 21 with the microprocessor based control system 22. The lipolysis device 21 may be sparingly energized by wall plugged power supply 23 or the battery operated power supply 24. The microprocessor system 22 controls the regulator 25 capable to supply the voltage adequate to the required level of the pads radiation 26 and for the required duration time of the session. Pads 26 energized by the regulator 25 provide the red light radiation of 635 nm wavelength required for the lipolysis process. The hour counter 29 counts the total time of the effective operation of the unit 21 with the LED pads 26 being switched on. The keyed activated momentary switch 27 provides the unit activation by the authorized personnel for duration of 20 minutes therapeutic radiation session. The indicator LED 28 indicates different stages of the session and special time periods after the end of the session as presented in the microprocessor program flow chart drawing and description.

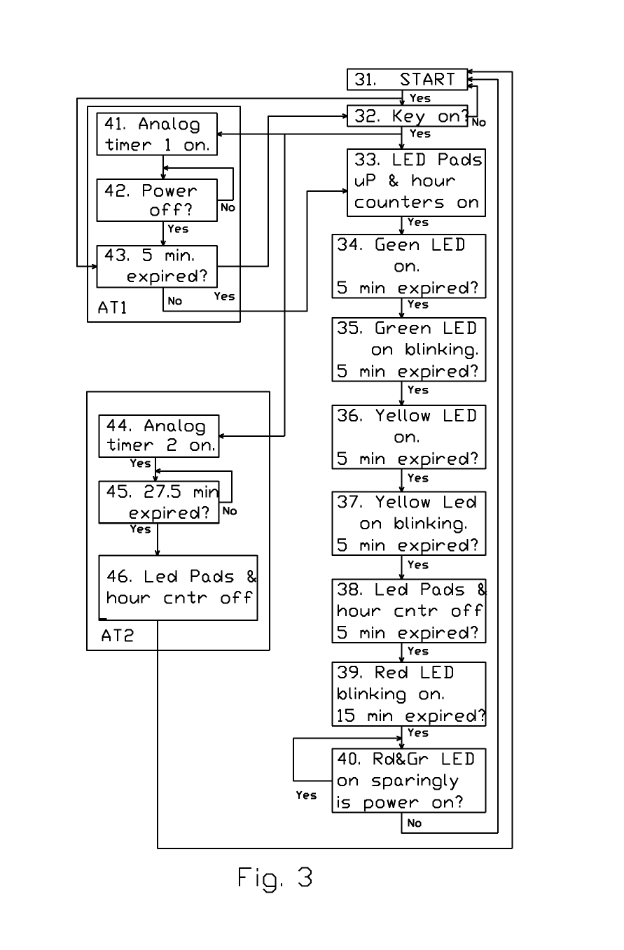

FIG. 3 shows the microprocessor program flow chart for the second embodiment of the lipolizis exercise device.

The program starts to run upon powering the unit in the block 31 and waits until the activation is performed by turning on the momentary key switch 27 (FIG. 2) in the block 32 and starts the digital timer provided by microprocessor and the hour meter in the block 33 and two additional analog timers AT1 in the block 41 and timer AT2 in the block 44. After the activation in the block 32 the therapeutic radiating LED pads 26 (FIG. 2) are switch on and the digital timer in the microprocessor starts to count the first 5 minutes of the 20 minutes radiation session in the block 34. Concurrently the analog timer AT2 in the block 44 is activated for approximately 27.5 min with 10% accuracy thus preventing the session to last longer than 30 min. (block 45 and 46) in case of the microprocessor failure. The microprocessor being the base of the microprocessor system 22 (FIG. 2) is programmed for the continuous 20 minutes session divided into four 5 minutes periods indicated by different modes of the bicolor red and green LED 28 (FIG. 2) indicated consequently by green, blinking green, yellow-orange and blinking yellow-orange colors of this LED 28 (FIG. 2) and performed in the blocks 34, 35, 36 and 37. This yellow-orange color of the LED 28 (FIG. 2) in last two modes are the effect of the red and green parts of this bicolor red and green LED 28 (FIG. 2) being switched on simultaneously. This indicated periods allow the customer to determine the stage of the session and estimate the time left to return the unit to the renting counter. Analog timer AT1 in the block 41 being activated concurrently with digital timer provided by the microprocessor and analog timer AT2 in the block 44 starts to count down first 5 minutes period of the 20 minutes session duration with the 10% accuracy. This counter allows the customer to take the unit being key activated on the counter and bring the unit with the disconnected power supply to the preferred training machine in the gym and power it again during this 5 minutes period (block 42 and 43) without the necessity of repeating the key activation, which is an important feature in case of using the wall plug type power supply 23 (FIG. 2) instead of the battery operated power supply 24 (FIG. 2). After the 20 minutes radiation session expires the therapeutic radiation pads 26 (FIG. 2) are off, hour counter is off and the indicator LED 28 (FIG. 2) turns red for 5 minutes indicating the end of the session. This is the time to return the unit back to counter without the additional charge. After this 5 minutes period the indicator LED 28 (FIG. 2) starts to blink red for 15 minutes (block 39) indicating the case, when the additional financial charge may be applied. After this 15 minutes period the indicator LED 28 (FIG. 2) will blink sparingly red and green (block 40). This indicates exceeding of the double period of time designated for the 20 minutes session and may be subject for a double charge for not returning the unit to the renting counter for such a long time. After the return the unit needs to be restarted by turning the keyed momentary activation switch 27 (FIG. 2). In the case of the battery operation the battery based power supply 24 (FIG. 2) has to be disconnected from the unit and connected again previously to the new key activation.

### CONCLUSION, RAMIFICATION AND SCOPE

The invention relates to the Lipolysis exercise device with the portability option and integrated tamper resistant hour counter for the device rental purpose. The particular embodiments has been illustrated and described in example only and is not intended to be limiting. It will be apparent to those skilled in art that various changes and modifications can be made without departing from the scope of the present invention. It is therefore to encompass within the appended claims all such changes and modifications that fall within the scope of the present invention.

What is claimed is:

1. A lipolysis device comprising:
a power supply,
a multitude of light radiating pads,
an hour counter.

2. The lipolysis device of claim 1 where said power supply is a wall plugged power supply.

3. The lipolysis device of claim 1 where said power supply is a battery operated power supply.

4. The lipolysis device of claim 1 capable to use said wall plugged power supply and said battery operated power supply sparingly.

5. The lipolysis device of claim 1 where said power supply is a regulated power supply capable of providing a multitude levels of radiation of said radiating pads.

6. The lipolysis device of claim 1 where said light radiating pads are based on light emitting diodes.

7. The lipolysis device of claim 1 where said light radiating pads are based on organic light emitting diodes.

8. The lipolysis device of claim 1 where said hour counter is a microprocessor based counter.

9. The lipolysis device of claim 1 where said hour counter is protected against tampering.

10. The lipolysis device of claim 1 further comprising a session timer capable of providing a required time of a radiation session.

11. The lipolysis device of claim 10 where said session timer is an electromechanical timer.

12. The lipolysis device of claim 10 where said session timer is an electronic timer.

13. The lipolysis device of claim 10 where said session timer is a microprocessor based timer.

14. The lipolysis device of claim 10 further comprising an authorization device like a lock, a keyed switch or keypad which has to be used in order to authorize each said radiation session.

15. The lipolysis device of claim 11 comprising a set of two said session timers connected in series in order to protect the patient from overexposure in case of a failure of one of said timers during said radiation session.

16. The lipolysis device of claim 15 further comprising a test indicators to test both said session timers before every said radiation session.

17. The lipolysis device of claim 13 further comprising an additional analog session timer running independently of said microprocessor based timer in order to prevent said patient said radiation overexposure in case of said microprocessor based counter failure.

18. The lipolysis device of claim 3, where said battery capacity would allow to energize said radiation pads for a shorter time than said time that could cause said patients said radiation overexposure.

19. The lipolysis device of claim 3 further comprising a timer allowing to replace said battery only in an early stage of said light radiation session, thus preventing said patient overexposure in case of the battery replacement during said session.

20. The lipolysis device of claim 10 further comprising an indicator capable to indicate a stage of said radiation session allowing a user to be able to return said lipolysis device after a rental session in a required period of time.
